(19) United States
(12) Patent Application Publication
(10) Pub. No.: US 2005/0059919 A1
(43) Pub. Date: Mar. 17, 2005

(54) Post-operative dressing for below knee amputees

(76) Inventor: Wallis Wiremu Toataua Farraday, Orlando, FL (US)

Correspondence Address: Steven B. Pokotilow, Esq.;Stroock & Stroock & Lavan LLP, 180 Maiden Lane, New York, NY, 10038, US

(21) Appl. No.: 10/789,228

(22) Filed: Feb. 26, 2004

Related U.S. Application Data
(60) Provisional application No. 60/450,816, filed on Feb. 27, 2003.

(51) Int. Cl. A61F 5/01; A61F 13/06; A61F 2/80

(52) U.S. Cl. 602/63; 623/36

(57) ABSTRACT

The present invention is directed toward a post-operative limb dressing and protection management system for below-the-knee amputees, preferably by providing a removable rigid dressing. The dressing of the present invention generally comprises a transparent thermo plastic liner that is covered by a removable rigid cast to provide relatively easy visual access to the limb.

### CROSS-REFERENCE TO RELATED APPLICATION

This application claims the benefit of U.S. Provisional Application Ser. No. 60/450,816, filed Feb. 27, 2003, entitled POST-OPERATIVE DRESSING FOR BELOW KNEE AMPUTEES, which is hereby incorporated herein by reference.

### FIELD OF THE INVENTION

The invention relates generally to a post-operative limb dressing for a below-the-knee amputee and, more particularly, to an immediate post-operative limb protection dressing and management system for below knee amputees comprising a unique post-operative rigid removable dressing. The present invention also related to a method of making such a dressing.

### BACKGROUND OF THE INVENTION

Various products and techniques exist in the market which are intended to protect the operative site of the residual limb, minimize edema, reduce pain and enhance healing time. Doctors commonly use soft dressings, conventional rigid dressings, immediate postoperative prostheses (IPOP), which is a rigid dressing with a temporary prosthetic, and removable rigid dressings (RRD) on transtibial amputations.

The soft dressings generally consist of sterile gauze and fluff and shrinker socks or elastic bandages to help control edema. These dressings, and the methods of applying them, are simple, require little time, can be practiced easily using many available materials and allow wound checks. However, there are many disadvantages of using a soft dressing, which include reduced edema control, increased risk of contractures, generation of high pressures that are detrimental to the skin, and the necessity of prolonged immobilization that can result in pulmonary complications and de-motivating depression.

The conventional rigid dressings and IPOP usually comprise a plaster cast that is applied in the operating room either immediately after surgery or within seven to ten days. A thigh-high cast is used to immobilize the knee joint and a supracondylar molding of the cast is used to prevent the cast from rotating or pistoning. Rigid dressings and IPOP are considered preferable because they can reduce pain and healing time, increase tolerance to weight bearing and enable early ambulation. However, the wound cannot be readily examined, which is a serious disadvantage to patients with vascular disease. Also, it is common for the patient to fall directly onto the end of the fresh amputation, exposing the amputation to infection, injury to the suture line and trauma to the soft tissue and bony extremity, which the conventional rigid dressing cannot prevent.

The RRD is made of plaster or fiberglass and is suspended by a stockinette and supracondylar cuff or sleeve. The cast does not extend to the thigh and can be slipped off like a transtibial socket, thereby allowing frequent observation while providing immobilization. A disadvantage of RRD is that is does not immobilize the knee and therefore there is a risk of knee flexion contractures and traction on the incision line caused by knee motion.

Accordingly, it is desirable to provide an immediate post-operative limb protection dressing, which overcomes shortcomings of existing dressings and techniques.

### SUMMARY OF THE INVENTION

Generally speaking, the present invention is directed towards a post-operative limb dressing and limb management system for below-the-knee amputees. In accordance with one aspect of the invention, a removable rigid dressing is provided for post-operative limb protection management. The dressing of the present invention generally comprises a transparent thermo plastic liner that is covered by a removable rigid cast. In a preferred embodiment, the cast also comprises an opening proximate the knee.

To form and apply the dressing, the rehabilitation team covers the residual limb with an elastic transparent thermo plastic gel liner, which allows the limb to be visually inspected while providing total contact hydrostatic pressure and continuously reduces the volume of the limb. The team then applies a distal pad at the distal end of the limb, and then pulls a sock or sock-like material over the limb. A spacer, such as a rubber tube, is applied over the sock, the spacer preferably spanning from the outer upper thigh to the distal end of the limb and to the inner upper thigh. A plastic wrap is used to wrap the limb, a gauze or other type of pad is placed on top of the knee, and a cast forming gauze is used to wrap the limb. The team shapes the residual muscle tissue. The cast is permitted to form and is then cut proximate the spacer to create a cast shell having top and bottom portions. The cast need not be cut along the entire periphery so long as the cast is removable. In a preferred embodiment the cast is cut in such a way so as to form a hinge portion proximate the distal end of the limb. A hole is preferably also cut in the cast proximate the gauze pad above the knee to create room and comfort for the knee. The spacer, gauze pad and plastic wrap are removed and the cast is placed on the limb again, held in place by one or more straps. Constructed as such, the cast and sock can be easily removed, and since the liner is transparent, the limb can be visually examined with ease.

Accordingly, it is an object of the invention to provide immediate post-operative limb protection dressing and management system.

It is another object of the invention to enhance residual limb definition and accelerate rehabilitation time.

Another object of the invention is to reduce distal socket migration.

Still another object of the invention is to reduce flexion contractures.

Another object of the invention is to protect the residual limb from trauma and negate external influence on healing.

A further object of the invention is to provide friction free comfort.

Yet another object of the invention is to provide total residual limb inspection during initial postoperative care.

Still another object of the invention is to reduce phantom pain.

Still other objects and advantages of the invention will in part be obvious and will in part be apparent from the specification and drawings.

The invention accordingly comprises the several steps and the relation of one or more of such steps with respect to each of the others, and the article possessing the features, properties, and the relation of elements, which are exemplified in the following detailed disclosure, and the scope of the invention will be indicated in the claims.

### BRIEF DESCRIPTION OF THE DRAWINGS

FIG. 1 is a side view of a post-operative limb after a liner and distal pad have been applied according to an embodiment of the invention.

FIG. 2 is a side view of a post-operative limb after a sock, a spacer, a plastic wrap and a gauze pad have been applied to the post-operative limb of FIG. 1.

FIG. 3 is a side view of a post-operative limb after a cast forming gauze has been wrapped around the post-operative limb of FIG. 2.

FIG. 4 is a perspective view of the cast of FIG. 3 in its open position after being cut along the perimeter.

FIG. 5 is a side view of a post-operative limb protected by a dressing according to an embodiment of the invention.

### DETAILED DESCRIPTION OF THE PREFERRED EMBODIMENTS

The invention relates generally to a post-operative limb dressing for a below-the-knee amputee and, more particularly, to an immediate post-operative limb protection dressing and management system for below knee amputees comprising a unique post-operative rigid removable dressing. The present invention also related to a method of making such a dressing.

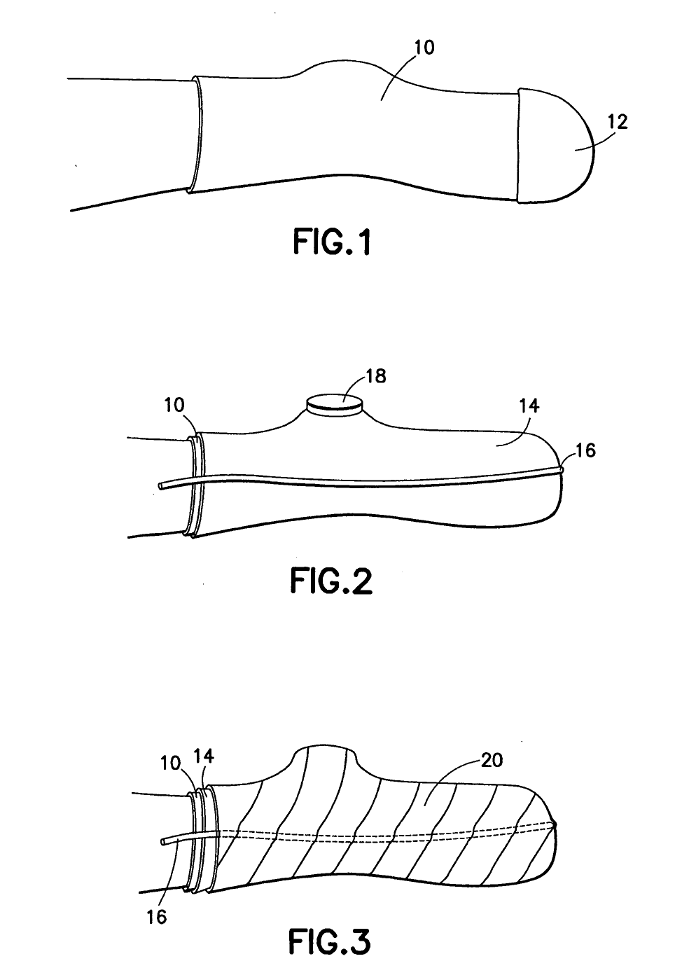

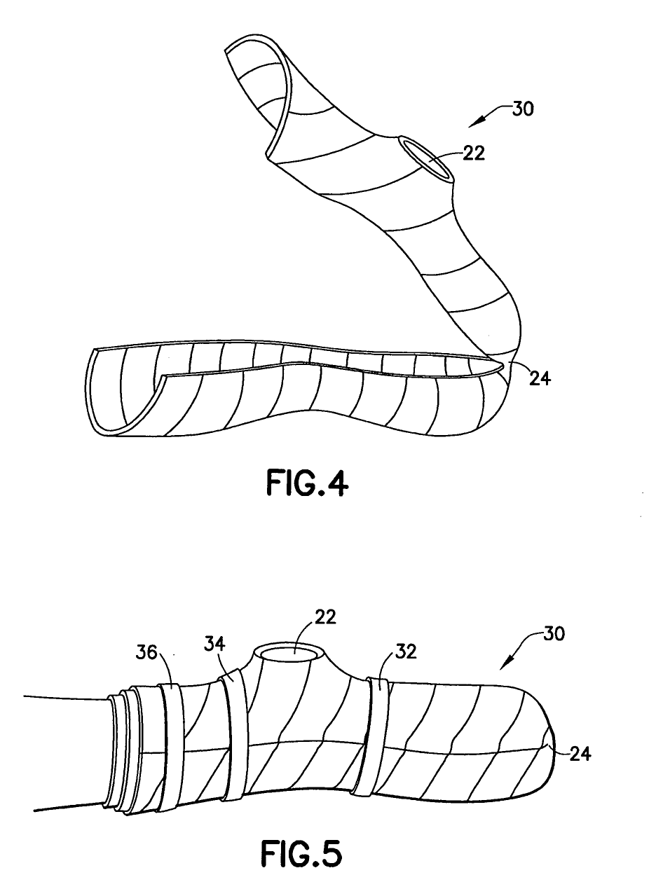

Generally speaking, the present invention is directed towards a post-operative limb dressing and limb management system for below-the-knee amputees. In accordance with one aspect of the invention as shown in FIGS. 1-5, a removable rigid dressing or cast 30 is provided for post-operative limb protection management. The dressing generally comprises a transparent thermo plastic gel liner 10 that is covered by a removable rigid cast 30. In a preferred embodiment, a sock 14 is placed over the gel liner 10, and cast 30 also comprises an opening 22 proximate the knee. One or more straps can be used to secure the cast in place to prevent movement while providing relatively easy removal. For highly vascular cases a patella opening can be made in the thermo plastic gel liner, and a small amount of silicone can be applied to the knee cap to lessen friction. Curlex frim the dressing is wrapped over the knee to prevent any window edema.

Referring to FIGS. 1-5, a preferred method of forming the dressing of the present invention will now be described. In a preferred embodiment of the invention, a below knee amputee is treated immediately subsequent to the amputation. The residual limb is covered with a protective material (such as conventional gauze) by the doctor after the amputation, which is not removed. An elastic, transparent thermo plastic gel liner 10 is then stretched and applied over the residual limb. In a preferred embodiment liner 10 is sprayed with silicone alcohol before being applied to the limb. Liner 10 is preferably tubular shaped, closed at one end and open at the opposite end. Liner 10 is applied to the limb via the open end, resulting in the distal end of the limb being fully covered by the closed end of the liner 10. Due to the transparency of liner 10, the limb can be examined through liner 10 without the need to remove liner 10 from the limb. Also, because liner 10 is elastic, it can be stretched to facilitate application and is formfitting. Liner 10 allows for total contact hydrostatic pressure, constantly reducing the volume of the residual limb. This consistent pressure provides for accelerated healing while reducing phantom pain.

In a preferred embodiment, as seen in FIG. 1, a distal pad 12 is applied to the distal end of the residual limb to provide extra protection of the distal end where the amputation took place. A sock or sock-like material 14 can then be placed over distal pad 12 and liner 10 to cover the residual limb.

As seen in FIG. 2, a spacer 16, preferably a rubber tube, is placed along the perimeter of the limb. The limb and spacer 16 are wrapped in a plastic wrap, thereby keeping spacer 16 in place and helping to prevent sock 14 from sticking to the cast that is to be applied. In a preferred embodiment, the plastic wrap is GLAD® Wrap sold by GLAD. A pad 18, preferably a gauze pad, of appropriate size and thickness is placed proximate the knee in order to mark the spot and provide more space for the knee once the cast forms.

As seen in FIG. 3, the residual limb is then wrapped with a conformable cast forming gauze 20, which is solidified to form a cast 30. I a preferred embodiment, before gauze 20 solidifies, the residual muscle tissue is shaped, preferably into predetermined zones, so as to better accept a prosthetis. Manipulating the residual muscle tissue into zones at this early stage helps to enhance rehabilitation and accelerate the maturity of the residual limb for weight bearing and non-weight bearing purposes. Manipulation of the tissue also aids in creating a total contact environment to reduce any potential forces on non-weight bearing tissue. Early intervention and manipulation and zoning of the tissue aids in accelerating rehabilitation time.

Once gauze 20 solidifies and forms cast 30, cast 30 is cut along the proximity of spacer 16, as depicted by dashed lines in FIG. 3. Spacer 16 provides guidance in cutting cast 30 into front and back halves while also providing a buffer to ensure that the inner layers, sock 14 and liner 10, are not cut. In a preferred embodiment, an opening 22 is also cut proximate or above the knee, where gauze pad 18 was previously placed, in order to provide for flexibility and comfort. Because cast 30 is cut along its periphery (either with or without a non-cut hinge portion 24 proximate the distal end of the limb), it is more easily removable from the limb.

The plastic wrap, spacer 16 and gauze pad 18 are all removed and cast 30 is repositioned and closed over sock 24 on the limb. One or more straps 32, 34, 36 can be used to secure cast 30. Preferably, strap 32 is placed at the distal end of the limb under the knee, strap 34 is placed directly above the knee, and strap 36 is placed at the top of cast 30. This ensures cast 30 will firmly remain on the limb while allowing easy removal.

The novel dressing, and dressing management system, of the present invention provides for improved post-operative limb protection management. It also enhances residual limb definition, accelerates rehabilitation time, reduces distal socket migration, reduces flexion contractures, and provides the patient with a feeling of security and sense of well being.

Although the dressing of the present invention is particularly suited for below knee amputees that have no potential for ambulating, as well as below knee amputees that are high risk to falling and damaging their limbs, the dressing is equally suited for any below the knee amputee.

It will be understood that while fundamental novel features of the invention as applied to preferred embodiments thereof have been described and pointed out, various omissions and substitutions and changes in the form and details of the disclosed invention may be made by those skilled in the art without departing from the spirit of the invention. For example, the term sock as used herein and in the claims should be interpreted in its broadest sense to be any material suitable for covering the gel liner and distal pad. In addition, the term rigid cast as used herein and in the claims should be interpreted in its broadest sense to be any material that is structurally suitable for protecting the residual limb. It is the intention, therefore, to be limited only as indicated by the scope of the claims appended hereto.

1. A removable post-operative limb dressing comprising:
a thermo plastic gel liner;
a protective covering over the liner;
a removable cast; and
a securing means for securing the removable cast onto the limb.

2. The dressing of claim 1, further comprising a distal pad, wherein the distal pad is placed at a distal end of the limb, over the thermo plastic gel liner and under the protective covering.

3. The dressing of claim 1, wherein the thermo plastic gel liner is tubular shaped and closed at one end and open at another end.

4. The dressing of claim 1, wherein the protective covering is a fabric.

5. The dressing of claim 1, wherein the protective covering is closed at one end and open at another end.

6. The dressing of claim 1, wherein the removable cast is cut along its periphery.

7. The dressing of claim 5, wherein the removable cast is cut into more than one portion.

8. The dressing of claim 1, wherein the removable cast is not completely cut and comprises a hinge keeping the removable cast as one piece.

9. The dressing of claim 1, wherein the one or more securing means comprise one or more straps.

10. The dressing of claim 1, wherein the cast comprises an opening proximate a joint.

11. The dressing of claim 1, wherein the dressing is suitable for a below-the-knee amputated leg.

12. A method of dressing a post-operative limb comprising:
applying a thermo plastic gel liner on a post-operative limb;
placing a distal pad on a distal end of the post-operative limb above the thermo plastic gel liner;
covering the post-operative limb with a protective covering over the thermo plastic gel liner;
wrapping the post-operative limb over the protective covering with a wrapping means;
placing a spacer around the perimeter of the limb prior to applying the cast forming gauze;
cutting the cast proximate the spacer;
wrapping the post-operative limb with a cast forming gauze, wherein the cast forming gauze solidifies to form a cast;
shaping the residual muscle tissue before the gauze solidifies;
cutting the cast along its periphery sufficient to permit removal of the cast; and
securing the cast on the post-operative limb with one or more securing means.

13. The method of claim 12, further comprising placing a pad proximate a joint and cutting the cast proximate the pad prior to securing the cast on the post-operative limb.

14. The method of claim 12, wherein the spacer is removed prior to securing the cast on the post-operative limb.

15. The method of claim 12, wherein cutting the cast along its periphery is cutting the cast into two separate portions.

16. A method of making a removable cast suitable for use with a post-operative limb comprising:
applying a thermo plastic gel liner on a post-operative limb;
covering the post-operative limb with a protective covering over the thermo plastic gel liner;
wrapping the post-operative limb over the protective covering with a wrapping means;
wrapping the post-operative limb with a cast forming gauze, wherein the cast forming gauze solidifies to form a cast; and
cutting the cast along its periphery sufficient to permit removal of the cast.

17. The method of claim 16, further comprising placing a pad proximate a joint and cutting the cast proximate the pad.

18. The method of claim 16, further comprising shaping the residual muscle tissue before the gauze solidifies.

19. The method of claim 16, further comprising placing a spacer around the perimeter of the limb prior to applying the cast forming gauze.

20. The method of claim 19, further comprising cutting the cast proximate the spacer.

21. The method of claim 19, wherein the spacer is removed prior to securing the cast on the post-operative limb.

22. The method of claim 16, wherein cutting the cast along its periphery is cutting the cast into two separate portions.
